(19) United States
(12) Patent Application Publication
(10) Pub. No.: US 2008/0208137 A1
(43) Pub. Date: Aug. 28, 2008

(54) Multi-Stage Sryinge and Methods of Using the Same

(76) Inventor: Frank M. Fago, Mason, OH (US)

Correspondence Address: Mallinckrodt Inc., 675 McDonnell Boulevard, HAZELWOOD, MO, 63042, US

(21) Appl. No.: 11/914,149

(22) PCT Filed: May 15, 2006
(86) PCT No.: PCT/US06/18806

§ 371 Date: Nov. 12, 2007

Related U.S. Application Data
(60) Provisional application No. 60/681,394, filed on May 16, 2005.

(51) Int. Cl. A61M 5/19; A61M 5/315

(52) U.S. Cl. 604/191; 604/236

(57) ABSTRACT

One or more aspects of the present invention relate to syringe having an intermediate plunger that includes a one-way valve having a fluid passage define therethrough. The syringe may be utilized to sequentially inject first and second medical fluids into a patient.

### CROSS REFERENCE TO RELATED APPLICATIONS

This application claims the benefit of U.S. Provisional Application No. 60/681,394, filed on 16 May 2006.

### FIELD OF THE INVENTION

The present invention relates to syringes, and more particularly to a syringe equipped with an intermediate plunger for enabling at least generally sequential delivery of first and second medical fluids.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Nuclear medicine utilizes radioactive material for diagnostic and therapeutic purposes by injecting a patient with a small dose of the radioactive material, which concentrates in certain organs or biological regions of the patient. Radioactive materials typically used for nuclear medicine include Technetium-99m, Indium-113m, and Strontium-87m among others. Some radioactive materials naturally concentrate toward a particular tissue, for example, iodine concentrates toward the thyroid. However, radioactive materials are often combined with a tagging or organ-seeking agent, which targets the radioactive material for the desired organ or biologic region of the patient. These radioactive materials alone or in combination with a tagging agent are typically referred to as radiopharmaceuticals in the field of nuclear medicine. At relatively lower doses of the radiopharmaceutical, a radiation imaging system (e.g., a gamma camera) provides an image of the organ or biological region that collects the radiopharmaceutical. Irregularities in the image are often indicative of a pathologic condition, such as cancer. Higher doses of the radiopharmaceutical may be used to deliver a therapeutic dose of radiation directly to the pathologic tissue, such as cancer cells.

In certain applications, multiple medical fluids may be injected into a patient. In positron emission tomography (PET) or single photon emission computed tomography (SPECT), a syringe may intake, contain, and subsequently inject a radioactive substance, such as a radiopharmaceutical. In magnetic resonance imaging (MRI), computed tomography (CT), radiography (e.g., x-ray), or ultrasound, a syringe may intake, contain, and subsequently inject a contrast agent. These applications also may utilize other medical fluids in combination, prior to, or after injecting the radiopharmaceutical or contrast agent. Unfortunately, these applications generally utilize multiple syringes or independent injection mechanisms, which can lead to time delays, dosing inaccuracies, a greater potential for contamination, a greater potential for fluid wastage, and other problems. For example, a significant quantity of the radiopharmaceutical may be left in a conventional syringe. In addition, the syringe utilized to administer the radiopharmaceutical may contain more residual radiopharmaceutical than desired, posing potential safety and/or disposal concerns.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A first aspect of the present invention is directed to a syringe having a plunger. This plunger includes a one-way valve having a fluid passage defined through an interior of the plunger to a downstream side of the plunger.

A second aspect of the present invention is directed to a flow control plunger having a check-valve disposed between an upstream fluid side and a downstream fluid side thereof. This check-valve includes an interior passage fluidly coupling the upstream and downstream fluid sides when the check-valve is in an open position.

Still third aspect of the invention is directed to a syringe barrel having a plunger check-valve actuator disposed inside the syringe barrel at a front portion of the syringe barrel.

Yet a fourth aspect of the invention is directed to a method of using a syringe. In particular, a flow control plunger disposed inside a syringe is actuated to enable fluid flow through an interior of the flow control plunger to a downstream side of the flow control plunger.

Still yet a fifth aspect of the invention is directed to a method of using a syringe. In particular, a plunger of the syringe is biased toward a terminus of the syringe to discharge a first medical fluid between the terminus and an intermediate plunger. The terminus of the syringe is contacted with the intermediate plunger, and a second medical fluid is discharged through the intermediate plunger while the terminus and the intermediate plunger are in contact.

Various refinements exist of the features noted above in relation to the various aspects of the present invention. Further features may also be incorporated in these various aspects as well. These refinements and additional features may exist individually or in any combination. For instance, various features discussed below in relation to one or more of the illustrated embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

The accompanying figures, which are included to provide further understanding of various aspects of the invention, illustrate exemplary embodiments of the present invention and, together with the description, serve to explain various principles of the invention.

FIG. 1 is a perspective view of an embodiment of what may be characterized as a multi-chamber, multi-stage, or sequential injection syringe having a first medical fluid in a front chamber thereof and a second medical fluid in a rear chamber thereof, the chambers separated by a intermediate flow control plunger of the syringe;

FIG. 2 is an enlarged side view of an embodiment of a body of the intermediate flow control plunger of FIG. 1;

FIG. 3 is an enlarged exploded view of an embodiment of the intermediate flow control plunger of FIGS. 1 and 2, illustrating an elastomeric piston cap exploded from the body;

FIG. 4 is an enlarged cross-sectional view of an embodiment of a terminal end portion of the multi-chamber, multi-stage, or sequential injection syringe and the intermediate flow control plunger of FIGS. 1-3, illustrating a check valve of the intermediate flow control plunger in a closed position;

FIG. 5 is an enlarged cross-sectional view of an embodiment of a terminal end portion of an embodiment of the multi-chamber, multi-stage, or sequential injection syringe and the intermediate flow control plunger of FIGS. 1-3, illustrating a check valve of the intermediate flow control plunger in an open position;

FIG. 6 is a cross-sectional view of an embodiment of a multi-chamber, multi-stage, or sequential injection syringe, illustrating the terminal end oriented substantially downward and the rear chamber being filled from an open end of a barrel of the syringe, with a pushrod withdrawn;

FIG. 7 is a cross-sectional view of an embodiment of the filled multi-chamber, multi-stage, or sequential injection syringe of FIG. 6, illustrating the terminal end oriented substantially upward to purge unwanted air from the rear chamber;

FIG. 8 is a cross-sectional view of an embodiment of a multi-chamber, multi-stage, or sequential injection syringe, illustrating a terminal end oriented substantially downward and the rear chamber being filled through a fill port in the barrel;

FIG. 9 is a cross-sectional view of an embodiment of the filled multi-chamber, multi-stage, or sequential injection syringe of FIG. 8, illustrating the terminal end oriented substantially upward to purge unwanted air from the rear chamber;

FIG. 10 is a cross-sectional view of an embodiment of a multi-chamber, multi-stage, or sequential injection syringe, illustrating a terminal end thereof pointing up and a needle inserted through a pushrod to fill the rear chamber with the second medical fluid;

FIG. 11 is a cross-sectional view of an embodiment of a multi-chamber, multi-stage, or sequential injection syringe, illustrating a check valve on the plunger of the pushrod and a check valve on the intermediate piston, the rear chamber being filled with the second medical fluid;

FIG. 12 is a cross-sectional view of an embodiment of a multi-chamber, multi-stage, or sequential injection syringe, illustrating an axial passageway through the pushrod with an open plunger, the rear chamber being filled with the medical fluid;

FIG. 13 is a cross-sectional view of an embodiment of a multi-chamber, multi-stage, or sequential injection syringe, illustrating another embodiment of the intermediate flow control plunger between first and second chambers;

FIG. 14 is a partial cross-sectional view of the multi-chamber, multi-stage, or sequential injection syringe of FIG. 13, further illustrating a first injection from the first chamber immediately prior to an injection transition or intermediate position of the intermediate flow control plunger between multiple injections of substances;

FIG. 15 is a partial cross-sectional view of an embodiment of the multi-chamber, multi-stage, or sequential injection syringe of FIG. 13, further illustrating a second injection from the second chamber directly through the intermediate flow control plunger immediately after the injection transition or intermediate position;

FIG. 16 is a flowchart illustrating an embodiment of a method of use or syringe preparation process utilizing one or more of the multi-chamber, multi-stage, or sequential injection syringes of FIGS. 1-15;

FIG. 17 is a flowchart illustrating an embodiment of a method of operation or imaging process utilizing one or more of the multi-chamber, multi-stage, or sequential injection syringes of FIGS. 1-15;

FIG. 18 is a flowchart illustrating an embodiment of a nuclear medicine process utilizing one or more of the multi-chamber, multi-stage, or sequential injection syringes of FIGS. 1-15;

FIG. 19 is a block diagram illustrating an embodiment of a radiopharmacy or system utilizing one or more of the multi-chamber, multi-stage, or sequential injection syringes of FIGS. 1-15; and

FIG. 20 is a block diagram illustrating an embodiment of a nuclear imaging system utilizing one or more of the multi-chamber, multi-stage, or sequential injection syringes of FIGS. 1-15.

### DETAILED DESCRIPTION OF SPECIFIC EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As discussed in detail below, various embodiments of the present invention include an intermediate flow control plunger for separating two medical fluids in a syringe, and also enabling sequential administration of the two fluids. Some disclosed embodiments of the intermediate flow control plunger substantially reduce or may even virtually eliminate the possibility of the two medical fluids mixing while within the syringe. A first medical fluid may be disposed in a first chamber generally downstream of the intermediate flow control plunger, while a second medical fluid may be disposed in a second chamber generally upstream of the intermediate flow control plunger. Thus, the first medical fluid may be injected downstream from the intermediate flow control plunger, and the second medical fluid may be sequentially injected by directly passing through the intermediate flow control plunger. For example, the intermediate flow control plunger may include a check valve, or one-way valve, or automatic valve mechanism that enables flow of the second medical fluid therethrough after the first medical fluid is at least substantially or entirely output from the syringe. Thus, the intermediate flow control plunger may generally prevent or substantially reduce the possibility of backflow of the first medical fluid from the first chamber to the second chamber, thereby substantially or entirely reducing a likelihood of internal mixing of the first and second medical fluids.

In one embodiment, an intermediate flow control plunger is used in a syringe for sequential delivery of two medical fluids. The intermediate flow control plunger may separate the syringe barrel into a front chamber that may contain a first medical fluid and a rear chamber that may contain a second medical fluid which may or may not be different from the first medical fluid. In one regard, the intermediate flow control plunger may be characterized as a check valve that substantially prohibits backflow from the front chamber to the rear chamber. In use, force from a pushrod of the syringe on the second medical fluid in the rear chamber causes the intermediate plunger to slide forward in the syringe barrel causing the first medical fluid in the front chamber to be discharged (e.g., out a nozzle of the syringe). The check valve may be designed to exhibit a high enough opening pressure to substantially reduce or prevent mixture of the two fluids during discharge of the first medical fluid. After the first medical fluid is discharged (e.g., administered to the patient), the force from the pushrod being biased toward the nozzle of the syringe may cause the intermediate plunger to contact a conical or at least generally tapered end of the syringe (by the nozzle), and force from the pushrod causes the check valve to open and allow the second medical fluid to pass through the intermediate plunger and be discharged from the syringe.

The various embodiments of the disclosed syringes, though not limited to nuclear medicine, may be particularly useful in some nuclear medicine procedures where a biocompatible flush (e.g., saline) may be used to flush a nozzle of the syringe, extension tubing interconnected with the syringe, and/or an injection site. Incidentally, a biocompatible flush may generally refer to any biocompatible fluid that does not significantly detrimentally affect the function of other compositions being administered by way of a syringe of the invention. Examples of appropriate biocompatible flushes include, but are not limited to, saline, sterilized water, heparin solution, and glucose solution.

For example, a single syringe can contain both a radiopharmaceutical and a biocompatible flush. Based on desired dosing parameters, a 5 mL syringe may be a suitable size for multi-stage syringes in nuclear medicine, although other sizes may be used for various injection applications. In general, the intermediate flow control plunger separates the first and second fluids in corresponding first and second chambers of a multi-stage syringe until injection. A syringe pushrod can be safely retracted before the injection to check for vein patency. With a single continuous push, the radiopharmaceutical may be injected first, and then the biocompatible flush (e.g., saline) may be injected afterwards. The biocompatible flush may be utilized to flush the radiopharmaceutical from the syringe and/or the injection site in one step (if desired) and/or may reduce the residual radiation in the syringe.

The benefits potentially provided by various embodiments of the invention may be numerous. For example, in some cases, there may be little or no need to purchase or stock biocompatible flush or an extra syringe and needle. In some cases, there may be no need to prepare a separate biocompatible flush syringe and needle. Another potential benefit is that various embodiments of the present invention may effectively enable only one injection to be performed that substantively includes what was previously two or more separate and distinct injections. Certain aspects of the invention may at least generally reduce chances of accidental needle sticks (e.g., due to utilizing one syringe instead of two). Other benefits of various aspects of the invention may potentially include one or more of the following: reduce need to dispose of saline vial and/or second syringe and needle; relieve need to draw saline into the syringe after the radiopharmaceutical injection to perform the syringe flush; reduced radiation exposure (e.g., due to the greater distance between the radiopharmaceutical and the user's hand and/or due to the flushing of the front end of the radiopharmaceutical syringe); fewer occurrences of drips and spills due to the handling of one syringe instead of two; and flushing may become so convenient that it may be used for procedures that normally do not get flushed.

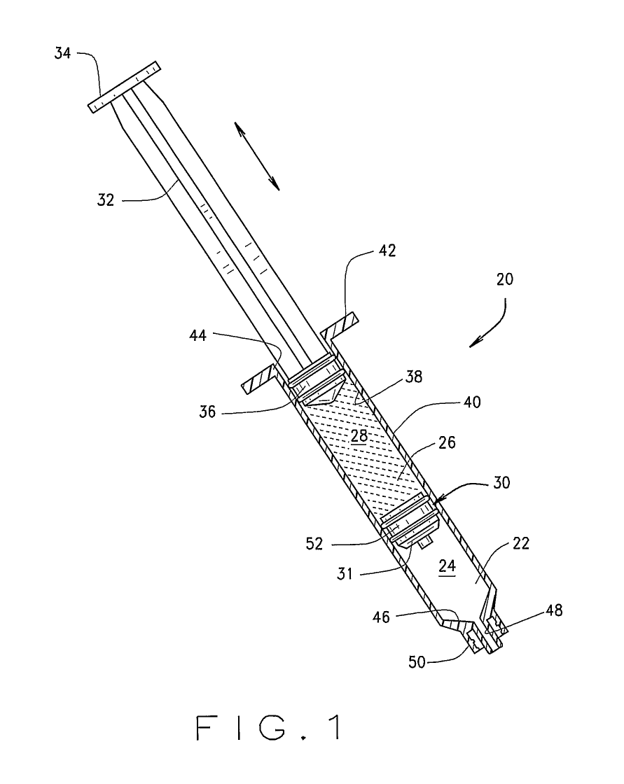

FIG. 1 is a perspective view of a multi-chamber, multi-stage, or sequential injection syringe 20 having a first medical fluid 22 disposed in a front chamber 24 of the syringe 20 and a second medical fluid 26 disposed in a rear chamber 28 of the syringe 20. The first medical fluid 22 may be any medical fluid appropriate for administration to a patient. Further, the second medical fluid may be the same as or different from the first medical fluid and may be any medical fluid appropriate for administration to a patient. For instance, in some embodiments, the first medical fluid may be a radiopharmaceutical or imaging contrast agent, and the second medical fluid may be a biocompatible flush. In addition, the embodiment of FIG. 1 may include a radioisotope generator, a fluid dispensing system, a power injector (e.g., motor, worm drive, radiation shield, etc.), a support structure, a rotatable arm (e.g., manual or robotic arm), a stand, an electronic control unit, a computer, an imaging system, a diagnostic system, or a combination thereof coupled to or generally associated with the syringe 20. In fact, each of the disclosed syringes may include one or more of these systems or components as discussed further below.

The front and rear chambers 24, 28 may be separated by an intermediate plunger 30, which includes a pressure activated check-valve 31. A pushrod 32 of the syringe 20 has an integral thumb tab 34 on one end and a plunger 36 (sometimes referred to as a proximal plunger) on the other end. The plunger 36 forms a seal with an inside wall 38 of a barrel 40 of the syringe 20. The intermediate plunger 30 may slide back and forth along the inside wall 38 of the barrel 40 in response to pressure from the front chamber 24 and/or pressure from the rear chamber 28 and therefore may be said to be “slidably positioned” in the barrel. The plunger 36 may also slide back and forth along the inside wall 38 of the barrel 40 as the pushrod 32 may be urged in and out (e.g., by a user or power injector). Accordingly, the plunger 36 may also be said to be “slidably positioned” in the barrel. It should be noted that some embodiments may not include an elongate pushrod 32 that is interconnected with the plunger 36. For instance, some embodiments for use with power injectors may include a plunger without an associated elongate pushrod 32. Further, the pushrod 32 may be generally utilized to bias or move the plunger 36; accordingly, any of a wide range of sizes, shapes, and designs of pushrods may be appropriate depending on the desired use of the syringe.

Finger grips 42 of the syringe 20 may be defined at an open end 44 of the barrel 40. At the end of the barrel 40 opposite the open end 44 is a terminus 46 of the barrel 40, which is sometimes referred to in the industry as a “conical end,” as shown, or it may be other shapes. A main passageway 48 may be defined in the terminus 46 of the syringe 20. The main passageway 48 may be bidirectional. In other words, the main passageway 48 may be designed to enable medical fluid to be both drawn into and discharged from the barrel 40 of the syringe 20 (e.g., in response to movement of the pushrod 32 and plunger 36). A luer fitting 50 or other appropriate interconnection device may also be formed on or attached to the syringe 20 (e.g., on or near the terminus 46).

To discharge the medical fluids 22, 26 from the syringe 20, pressure may be applied to the thumb tab 34 of the pushrod 32 causing the plunger 36 to slide down the inside wall 38 of the barrel 40, at least generally pressurizing the second fluid 26 in the rear chamber 28. The pressure from the second fluid 28 may act on the intermediate plunger 30 causing the intermediate plunger 30 to slide down the inside wall 38 of the barrel 40 to apply pressure on the first medical fluid 22. As the plunger 36 on the pushrod 32 and the intermediate plunger 30 slide down the barrel 40, the check-valve 31 in the intermediate plunger 30 may be closed which keeps the second medical fluid 26 in the rear chamber 28 separate from the first medical fluid 22 in the front chamber 24 during discharge of the first medical fluid 22. As the plunger 36 and the intermediate plunger 30 continue sliding down the inside wall 38 of the barrel 40, the first medical fluid 22 may be discharged through the main passageway 48 (e.g., for administration to a patient). After substantially all of the first medical fluid 22 has been discharged from the syringe 20, the intermediate plunger 30 makes contact with the terminus 46 of the syringe 20. When the intermediate plunger 30 contacts the terminus 46, continued pressure applied to the tab 34 of the pushrod 32 causes the plunger 36 (which is attached to the pushrod 32) to slide down the inside wall 38 of the barrel 30, increasing the pressure on the second medical fluid 26, which causes the check valve 31 of the intermediate plunger 30 to open allowing the second medical fluid 26 to pass through the check valve 31 into the main passageway 48 for discharge from the syringe 20.

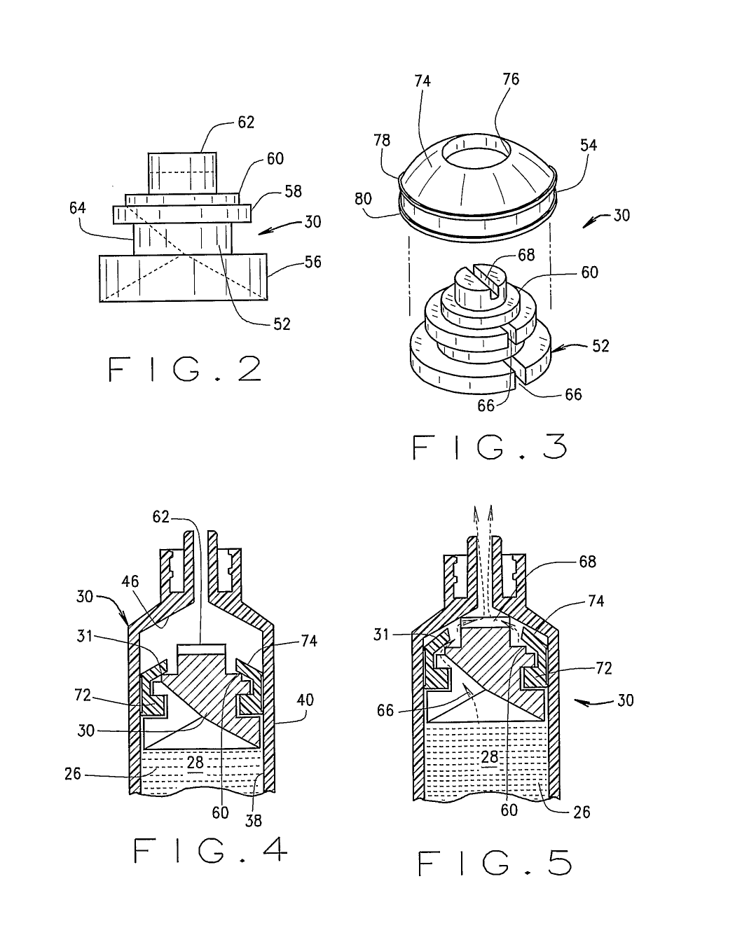

Referring now to FIGS. 2 and 3, the intermediate plunger 30 includes a body 52 and a flexible, resilient, or elastomeric piston cap 54. The body 52 of the intermediate plunger 30 includes a rear flange 56, a forward flange 58, a circumferential seat 60, and a protruding nose 62. The rear flange 56 and the forward flange 58 define a retention channel 64 therebetween to accommodate a retention ring 72 (FIGS. 4 and 5) of the elastomeric piston cap 54. The retention ring 72, when disposed between the rear and forward flanges 56, 58 of the body 52, may function to at least assist in holding the elastomeric piston cap 54 on the body 52 of the intermediate plunger 30. Incidentally, the flanges 56, 58 and retention ring 72 may be one manner of providing an appropriate interconnection between the body 52 and the elastomeric piston cap 54; other manners of providing an appropriate interconnection may be utilized. Further, the intermediate plunger 30 illustrates one embodiment of intermediate plungers. It should be noted that other intermediate plungers than enable fluid to flow through the intermediate plunger upon the intermediate plunger contacting the terminus of the syringe are within the scope of the disclosed embodiments.

Referring to FIG. 3, an intermediate passageway 66 may be defined in each of the rear flange 56 and the forward flange 58 of the body 52 of the intermediate plunger 30. Further, a nose passageway 68 may be defined in the nose 62 of the body 52. The elastomeric piston cap 54 includes a flexible lip 74 which has an aperture 76 (e.g., centrally located) defined therein. Around an outer circumference of the elastomeric piston cap 54 is a first circumferential seal 78 and a second circumferential seal 80 which seal against the inside wall 38 of the barrel 40 of the syringe 20. While the elastomeric piston cap 54 is shown as having first and second seals 78, 80, other embodiments of the elastomeric piston cap 54 may additionally and/or alternatively include other sealing features to promote a seal between the elastomeric piston cap 54 and the inside wall 38 of the barrel 40 of the syringe 20.

Referring to FIGS. 4 and 5, the flexible lip 74 of the intermediate plunger 30 may interface with the seat 60 to provide a fluid seal between the two components. In FIG. 4, the check valve 31 is closed, and in FIG. 5, the check valve 31 is open. When the check valve 31 is closed, the second medical fluid 26 may be confined in the rear chamber 28 between the intermediate plunger 30 and the plunger 36. When the check valve 31 is open, the second medical fluid 26 may be discharged from the syringe 20 as indicated by the dashed flow arrows shown in FIG. 5. As previously mentioned, the check valve 31 may be opened by pressure. When the check valve 31 is open, the second medical fluid 26 may flow from the rear chamber 28 through the intermediate passageway 66, past the seat 60, through the aperture 76 in the lip 74 of the elastomeric piston cap 54, and through the nose passageway 68 into the main passageway 48 of the syringe 20.

As the intermediate plunger 30 travels towards the terminus 46 of the barrel 40 while the first medical fluid 22 from the front chamber 24 is being discharged, the check valve 31 is in the closed position as shown in FIG. 4. When the first medical fluid 22 has been substantially discharged, the nose 62 of the intermediate plunger 30 contacts the terminus 46 of the barrel 40 causing the intermediate plunger 30 to stop advancing toward the main passageway 68 of the syringe 20. As continued pressure is exerted on the pushrod 32, the pressure of the second medical fluid 26 increases because it is trapped in the second chamber 28 between the plunger 36 and the intermediate plunger 30, and the check valve 31 is closed so the second fluid 26 at least temporarily has no place to go. As the pressure of the second medical fluid 26 reaches a “cracking pressure”, the check valve 31 opens as shown in FIG. 5, allowing the second medical fluid 26 to be discharged from the syringe 20 as indicated by the flow arrows (FIG. 5). A seal pressure and the cracking pressure may be independent and can be tuned for best performance. The “seal pressure” generally refers to the force that the first circumferential seal 78 and the second circumferential seal 80 apply on the inside wall 38 of the syringe barrel 40. The seal pressure may be relatively light, so friction between the seals 78, 80 and the inside wall 38 does not cause the intermediate plunger 30 to stick in the barrel 40. In addition, the cracking pressure can be set high enough to effectively promote the intermediate plunger 30 overcoming the friction. This facilitates the ability of the intermediate plunger 30 to be pushed toward the terminus 46 of the syringe 20 in order to allow the second medical fluid 26 to be discharged. The cracking pressure may be characterized as a function of the diameter of the elastomeric piston cap 54 on the intermediate plunger 30.

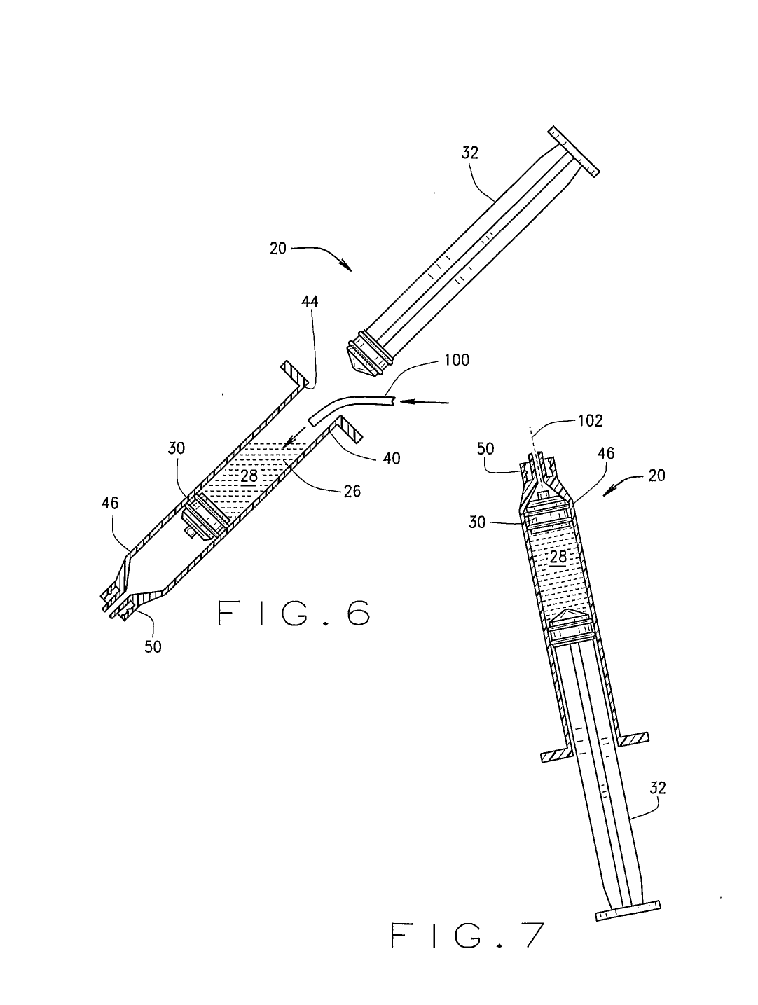

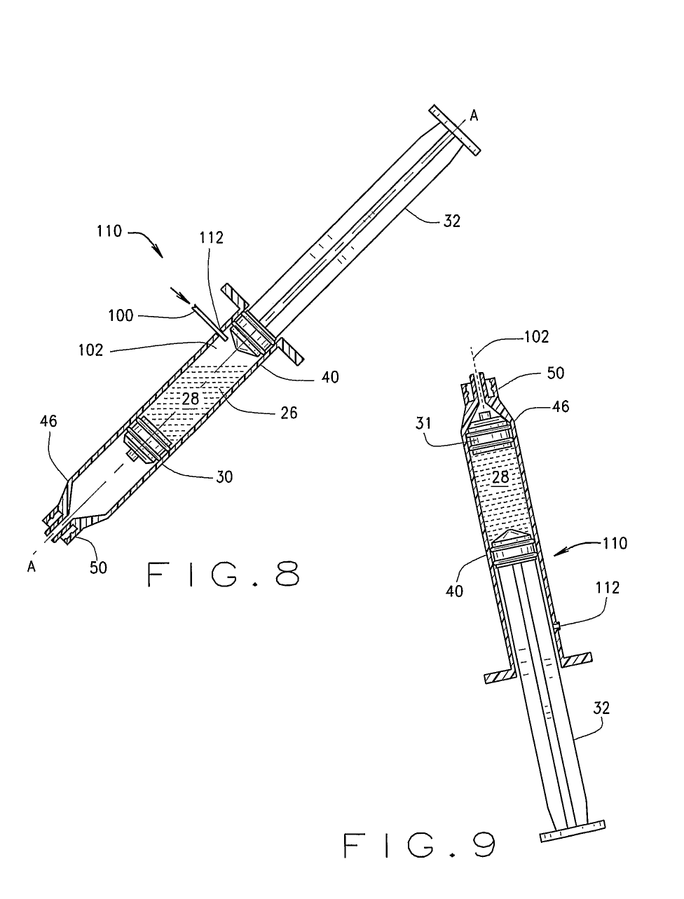

FIG. 6 is a cross-sectional view of a syringe 20 with the terminus 46 pointing downward and the rear chamber 28 being filled via the open end 44 of the barrel 40, with the pushrod 32 dissociated from the barrel 40. When using this filling technique, the intermediate plunger 30 may be positioned away from the terminus 46 in order to purge air from the syringe 20 which will be described in greater detail below. As shown in FIG. 6, a fill tube 100 may be inserted in the open end 44 of the barrel 40, and the second fluid flows from a source through the fill tube 100 and into the rear chamber 28. When the rear chamber 28 is filled with the desired amount of the second medical fluid 26 or more than actually may be desired, the pushrod 32 may be inserted into the open end 44 of the barrel 40. This process may result in trapping some unwanted air in the second chamber 28 (which may be purged from the syringe 20). FIG. 7 is a cross-sectional view of the filled syringe 20 from FIG. 6 after filling with the second medical fluid 26. The terminus 46 may be pointing upward to purge trapped air 102 from the rear chamber 28. After the syringe 20 has been inverted from the orientation of FIG. 6, the pushrod 32 may be depressed to force the intermediate plunger 30 into contact with the terminus 46 of the syringe 20. As more pressure is applied to the pushrod 32, the check valve 31 opens, allowing any trapped air 102 in the rear chamber 28 to be discharged from the syringe 20 through the same flow path as described above for the second medical fluid 26. After the unwanted air has been discharged, the syringe may be described as “pre-filled” with the second medical fluid. The “prefilled syringe” of FIG. 7 may be sold or shipped, as is, to allow a user to fill the front chamber with a first medical fluid which may have a short shelf life, such as a radiopharmaceutical. A “pre-filled” syringe may be prefilled with either one or two medical fluids.

The front chamber 24 may be filled using conventional techniques. The luer fitting 50 may be elevated and connected to a source of the first medical fluid 22. The user simply pulls back on the push-rod 32, like conventional syringes. When the push-rod 32 is drawn away from the terminus 46 and toward the open end 44 of the barrel 40, the check valve 31 remains closed and the intermediate plunger 30 slides away from the terminus 46, drawing the first medical fluid 22 into the front chamber 24. The luer fitting 50 may then be disconnected, and any unwanted air may be purged from the from the front chamber 24 using conventional techniques. When provided to end users having medical fluids in both the front chamber and the rear chamber, the syringe may be said to be “prefilled” with a plurality (e.g., two) medical fluids.

FIG. 8 is a cross-sectional view of a multi-chamber, multi-stage, or sequential injection syringe 110 with a fill port 112 defined in the barrel 40. All the other components of the syringe 110 may be at least similar to that of the syringe 20 of FIG. 1, and accordingly, the generally corresponding components will be referred to using the same identification numbers. In FIG. 8, the terminus 46 may be pointing downward to facilitate filling of the rear chamber 28 through a fill port 112 with the second medical fluid 26. The pushrod 32 has been withdrawn on one side of the fill port 112, and the intermediate plunger 30 has been positioned on the other side of the fill port 112 to enable a filling process. A fill tube 100 may be inserted into the fill port 112, and the second medical fluid 26 flows from a source through the fill tube 100 and into the rear chamber 28 of the syringe 30. The syringe 110 defines an axis as indicated by the line A-A. The orientation of the fill port 112 and the fill tube 100 may be generally normal or at least non-parallel to the axis A-A. Some air 102 may be trapped in the rear chamber 28. Subsequent to a desired amount of the second medical fluid being disposed in the rear chamber 28, the fill tube 100 may be withdrawn from the fill port 112, and the pushrod 32 may be depressed until the plunger 36 interfaces with a portion of the inside wall 38 of the barrel 40 between the fill port 112 and the terminus 46.

FIG. 9 is a cross-sectional view of the syringe 110 of FIG. 8 after filling with the second medical fluid 26, with the terminus 46 pointing upward to purge air 102 from the rear chamber 28. After the syringe 110 has been inverted from the orientation of FIG. 8, the pushrod 32 may be depressed to force the intermediate plunger 30 into contact with the conical end or terminus 46. Upon sufficient pressure being applied to the pushrod 32, the check valve 31 opens, allowing trapped air 102 in the rear chamber 28 to be discharged from the syringe 110 through the substantially same flow path as described above for the second medical fluid 26.

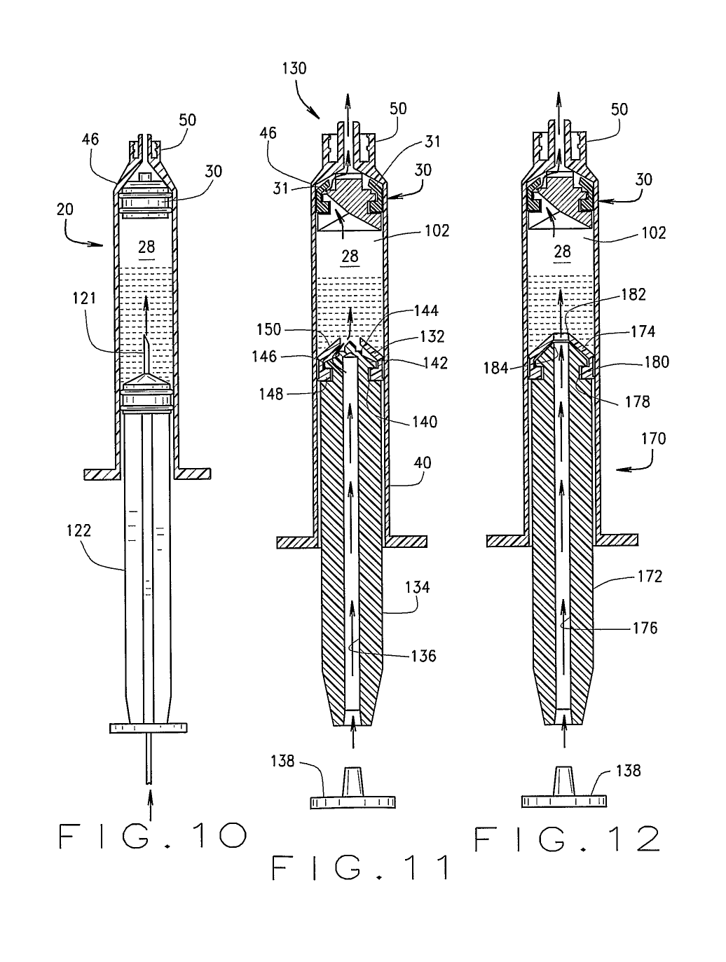

FIG. 10 is a cross-sectional view of a syringe 120. Similar components to the syringe 20 of FIG. 1 are referred to using the same numerals; different components have been assigned new identification numbers. The terminus 46 may be pointing up, and a fill needle 121 has been inserted through an axial passageway in a pushrod 122 of the syringe 120, to fill the rear chamber 28 with the second medical fluid 26. The fill needle 121 extends through the plunger 36 which may reseal after the fill needle 121 has been withdrawn. The second medical fluid 26 flows from a source, through the fill needle 121, and into the rear chamber 28 of the syringe 120. Subsequent to a desired amount of the second medical fluid 26 being disposed in the rear chamber 28, the fill needle 121 may be withdrawn from the pushrod 122. The syringe 120 may be positioned in any of a number of appropriate orientations during the fill process. However, at some point in the fill process, the terminus 46 of the syringe 120 may be elevated in order to purge unwanted air and/or bubbles from the rear chamber 28. If air 102 is trapped in the rear chamber 28, it may be purged by further depressing the modified pushrod 122 to expel the air 102 from the syringe through the substantially same flow path that the second medical fluid 26 travels to exit the syringe 120, previously described.

FIG. 11 is a cross-sectional view of another multi-chamber, multi-stage, or sequential injection syringe 130 having a second check valve 132 on a plunger of a pushrod 134 and the previously described check valve 31 on the intermediate plunger 30. In this view, the second check valve 132 is shown in the open position, and the syringe 130 is being filled. Similar components to the syringe 20 of FIG. 1 are referred to with identical numbers, and new components have been assigned new numbers. In this figure, the intermediate plunger 30 may be in contact with the conical end or terminus 46. The pushrod 134 may be in the barrel 40 and may be separated from the intermediate plunger 30 to define the rear chamber 28. The pushrod 134 has an axial passageway 136 defined therein that may be sealed on one end by a removable thumb tab 138 (or other appropriate sealant or sealing device) and the second check valve 132. In this view, the thumb tab 138 may be disengaged from the pushrod 134.

To fill this syringe 130, the second medical fluid 26 from a source may be introduced through the axial passageway 136, flows through the second check valve 132, and flows into the rear chamber 28 as indicated by the flow arrows of FIG. 11. Subsequent to a desired amount of the second medical fluid 26 being disposed in the rear chamber 28, the thumb tab 138 may be inserted in the axial passageway 136 to seal the second medical fluid 26 in the axial passageway 136. On the end of the pushrod 134 opposite the removable thumb tab 138, the pushrod 134 includes a pushrod retention channel 140, pushrod flow passageways 142 in fluid communication with the axial passageway 136, and a seat 144. A pushrod elastomeric cap 146 includes a pushrod retention ring 148 sized to engage the pushrod retention channel 140 to removably attach the cap 146 to the pushrod 134. The pushrod elastomeric cap 146 also includes a flexible lip 150 to engage the seat 144 which form the main components of the second check valve 132. In this figure, the check valve may be open so the lip 150 does not touch the seat 144. When the check valve is closed, the lip 150 may engage the seat 144 blocking the flow of fluid from the rear chamber 28 back up the axial passageway 136 of the pushrod 134. During the fill process, the syringe 130 may be in any number of appropriate orientations.

Like the other fill processes discussed herein, there may be bubbles and/or unwanted air 102 trapped in the rear chamber 28. To purge the unwanted air from the rear chamber 28, the terminus 46 may be oriented at least generally upward, and the pushrod 134 may be pushed further into the barrel 40, which expels the unwanted air 102 as previously described in FIGS. 6-9.

FIG. 12 is a cross-sectional view of a multi-chamber, multi-stage, or sequential injection syringe 170 having a pushrod 172 with an axial passageway 176 defined therein, and an open plunger 174. In this view, the rear chamber 28 is being filled with the second medical fluid 26. Similar components are identified with the same numbers as the syringe in FIG. 1, and different components have been assigned new numbers. The axial passageway 176 defined in the pushrod 172 may be open on both ends. One end of the pushrod 172 may be designed to receive a removable thumb cap 138 or other appropriate sealing device/material, and the other end has the open plunger 174 attached thereto. In this figure, the thumb cap 138 may be removed from the pushrod 172. The end of the pushrod 172 that carries the open plunger 174 forms a pushrod retention channel 178 that engages a retention ring 180 on the open plunger 174. The open plunger 174 defines a flow outlet 182 that may be in fluid communication with an outlet port 184 of the axial passageway 176 in the pushrod 172.

As shown by the flow arrows in FIG. 12, the second medical fluid 26 flows from a source through the axial passageway 176 in the pushrod 172, through the outlet port 184 and the flow outlet 182 in the open plunger 174 into the rear chamber 28. During the fill process, the syringe may be in any of a number of appropriate orientations.

When filling is complete, the thumb cap 138 may be engaged in the axial passageway 176 of the pushrod 172. This may leave unwanted bubbles and/or air 102 in the rear chamber 28. To clear the syringe 170 of unwanted air, the terminus 46 may be elevated, and the pushrod 172 may then be depressed further to discharge the unwanted air 102 from the syringe 170, as indicated by the flow arrows. Again, the flow path of the unwanted air 102 may be the same as the second medical fluid 26 through the check valve 31 and the intermediate plunger 30.

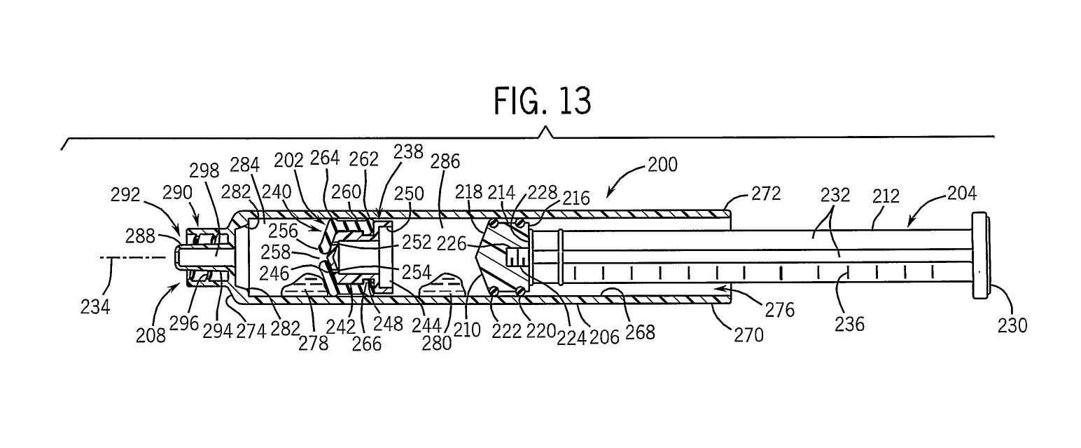

FIGS. 13-15 illustrate another embodiment of a syringe 200. FIG. 13 is a cross-sectional view of the syringe 200, illustrating an alternative embodiment of an intermediate flow control plunger or flow through valve plunger 202. In the illustrated embodiment of FIG. 13, the syringe 200 may include a primary plunger 204 and an elongated fluid container or syringe barrel 206 having an external fluid coupling such as a luer fitting 208. The luer fitting 208 may be coupled to a variety of fluid exchange or delivery systems, which may include tubing, valves, gravity fed containers, power injectors, electronic controls, injection needles, and so forth. In addition, the embodiment of FIGS. 13-15 may be coupled to or generally associated with a radioisotope generator, a fluid dispensing system, a power injector (e.g., motor, worm drive, radiation shield, etc.), a support structure, a rotatable arm, a stand, an electronic control unit, a computer, an imaging system, a diagnostic system, or a combination thereof.

The primary plunger 204 includes a primary plunger head 210 coupled to a pushrod 212. For example, the primary plunger head 210 may be removably coupled to the pushrod 212 via a variety of fastening mechanisms, such as mating threads, snap fit mechanisms, compression fit mechanisms, or various tool free fasteners. In the illustrated embodiment, the primary plunger head 210 may include a generally cylindrical body 214 having a flat side 216 and an opposite curved or conical side 218. In addition, the primary plunger head 210 may include one or more outer seals, such as a plurality of sequential o-rings 220 and 222, disposed about the generally cylindrical body 214. The primary plunger head 210 may include a removable fastening mechanism, such as an internally threaded member or female threads 224 extending inward from the flat side 216. Similarly, the pushrod 212 may include a removable fastening mechanism, such as an externally threaded member or male threads 226, extending outwardly from a flat side 228. Thus, the primary plunger head 210 may be removably coupled to the pushrod 212 by rotatingly driving the male threads 226 into the female threads 224 until the flat sides 216 and 228 may be generally flush with one another. In addition, the pushrod 212 may include an end member 230 disposed on an opposite end from the male threads 226. Similar to the embodiment of FIGS. 1-12, the pushrod 212 may include a plurality of lengthwise ribs 232, such as a set of four lengthwise ribs, arranged symmetrically about a lengthwise or central axis 234 of the primary plunger 204. A plurality of measurement indicia 236 may be disposed along the length of the pushrod 212 in a generally sequential offset arrangement.

As mentioned above, the syringe 200 of FIG. 13 may include one or more floating valve plungers or intermediate flow control plungers, such as the intermediate flow control plunger 202. In certain embodiments, the intermediate flow control plunger 202 may include a generally central, internal, or flow through check valve. In other words, the intermediate flow control plunger 202 may be configured to enable fluid to pass directly through rather than around the intermediate flow control plunger 202 in response to a pressure differential between opposite sides of the intermediate flow control plunger 202. In the illustrated embodiment, the intermediate flow control plunger 202 may include a fluid passage plunger insert 238 and a flexible plunger sleeve 240. In certain embodiments, the flexible plunger sleeve 240 may include a resilient, elastomeric, or generally flexible material, while the fluid passage plunger insert 238 may be generally rigid. In addition, the fluid passage plunger 238 and the flexible plunger sleeve 240 may have generally circular or annular geometries, which may be disposed concentrically with respect to one another. Also, the intermediate flow control plunger 202 may have a continuous outer seal, such as one or more o-rings, as discussed in further detail below.

The illustrated fluid passage plunger insert 238 may include a generally cylindrical body portion 242 having an open end 244 and an opposing throat end 246. In addition, the generally cylindrical body portion 242 may include an annular groove 248 and a protruding flange portion 250 disposed adjacent the open end 244. The throat end 246 may have a generally tapered, inwardly angled, or conical geometry, which includes one or more fluid passages. For example, the throat end 246 may include axially offset passages 252, 254, which may be normally closed or sealed by the flexible plunger sleeve 240. In certain embodiments, the throat end 246 may include fewer or greater numbers of passages, such as 1, 3, 4, 5, 6, 7, 8, 9, 10, or more. These passages, e.g., 252, 254, enable fluid to flow directly through the interior of the intermediate flow control plunger 202, rather than around the periphery of the intermediate flow control plunger 202 at the seal interface with the syringe barrel 206. As illustrated, the axially offset passages 252, 254 may be substantially covered and sealed by a flexible mouth portion 256 of the flexible plunger sleeve 240. In other words, the flexible mouth portion 256 may be substantially or mostly closed across the throat end 246 of the fluid passage plunger insert 238 except for an opening therein (e.g., axial opening 258). As illustrated, the axial opening 258 may be disposed along the central axis 234, whereas the axially offset passages 252, 254 may be disposed at a substantial distance or offset from the central axis 234.

The flexible plunger sleeve 240 includes a generally cylindrical body 260 having a plurality of annular outer seals (e.g., o-ring portions 262, 264) and a generally annular latch portion 266. In the illustrated embodiment, the generally cylindrical body 260 of the flexible plunger sleeve 240 may be disposed concentrically about the generally cylindrical body portion 242 of the fluid passage plunger insert 238, such that the latch portion 266 may extend removably into the annular groove 248. As such, the fluid passage plunger insert 238 may be removably coupled or snap fit with the flexible plunger sleeve 240, such that the intermediate flow control plunger 202 may be disassembled, cleaned, and reused if desirable.

In certain embodiments, the fluid passage plunger insert 238 may be molded, machined, or generally manufactured with a variety of generally rigid materials, e.g., plastic. The flexible plunger sleeve 240 may be molded or generally manufactured from a variety of flexible or resilient materials, such as rubber. As discussed in further detail below, the fluid passage plunger insert 238 cooperates with the flexible plunger sleeve 240 to at least substantially or entirely separate fluids disposed on opposite sides of the intermediate flow control plunger 202. Upon reaching or passing a certain pressure differential between opposite sides of the intermediate flow control plunger 202, the flexible plunger sleeve 240 may enable fluid flow directly through an interior of the fluid passage plunger insert 238 rather than around the periphery of the intermediate flow control plunger 202.

As further illustrated in FIG. 13, the syringe barrel 206 includes an interior surface 268 defining a generally cylindrical passageway and an exterior surface 270 exhibiting a generally cylindrical geometry 270, which both extend lengthwise along the syringe barrel 206 between a first end 272 and a second end 274 thereof. In certain applications, one or more of the intermediate flow control plungers 202 and the primary plunger 204 may be disposed lengthwise along the interior surface 268 through an opening 276 at the first end 272 of the barrel 206. The plungers 202 and 204 may be offset from one another and from the second end 274 of the barrel 206 to accommodate two or more substances or fluids. For example, a first medical fluid 278 may be disposed between the intermediate flow control plunger 202 and the second end 274 of the barrel 206. In addition, a second medical fluid 280 may be disposed between the primary plunger head 210 and the second intermediate flow control plunger 202. In certain embodiments, the first medical fluid 278 may include a radiopharmaceutical, a contrast agent, a drug, or a combination thereof. By further example, the second medical fluid 280 may include a biocompatible flushing or cleaning substance, such as a heparin solution, sterilized water, a glucose solution, saline, or another suitable substance. The interspacing between the one or more secondary floating valve plungers or intermediate flow control plungers 202, the primary plunger head 210, and the second end 274 of the barrel 206 may depend on the volume, quantity, or dose of the first medical fluid 278, the second medical fluid 280, and so forth.

The syringe barrel 206 may include a flow control actuator 282 extending inwardly (e.g., toward the axis 234) from the interior surface 268 of the barrel 206 near the second end 274 thereof. As discussed in further detail below, the flow control actuator 282 may engage the outer periphery of the intermediate flow control plunger 202, such that the flexible mouth portion 256 may be forced forward away from the throat end 246 to enable injection or general flow of the second medical fluid 234. In other words, the first medical fluid 278 disposed in a first chamber 284 may be forced outwardly through the luer fitting 208 in response to forward movement of the intermediate flow control plunger 202. Upon reaching the flow control actuator 282, the flexible plunger sleeve 240 of the intermediate flow control plunger 202 opens in a forward direction to enable the second medical fluid 280 disposed in a second chamber 286 to flow directly through the interior of the intermediate flow control plunger 202 in response to axial movement of the primary plunger 204. Thus, the flow control actuator 282 may be described or defined as a plunger check valve actuator, which triggers or actuates the transition of the check valve 240 from a generally closed position to an open position enabling flow through the interior of the intermediate flow control plunger 202.

In the illustrated embodiment, the luer fitting 208 may include a male luer 288 and a luer collar 290. For example, the luer collar 290 may be disposed concentrically about the male luer 288, such that these components 288, 290 define an interspace 292 having one or more removable fastening mechanisms. By further example, the male luer 288 may include a compression fitting or tapered or external surface 294, while the luer collar 290 may include internal threads 296. In certain embodiments, the luer fitting 208 may include a flow control mechanism (e.g., a manual or electronic valve) to open and close the fluid flow relative to the syringe 200. The luer fitting 208 may include a generally central fluid flow passage 298 extending through the male luer 288 along the axis 234.

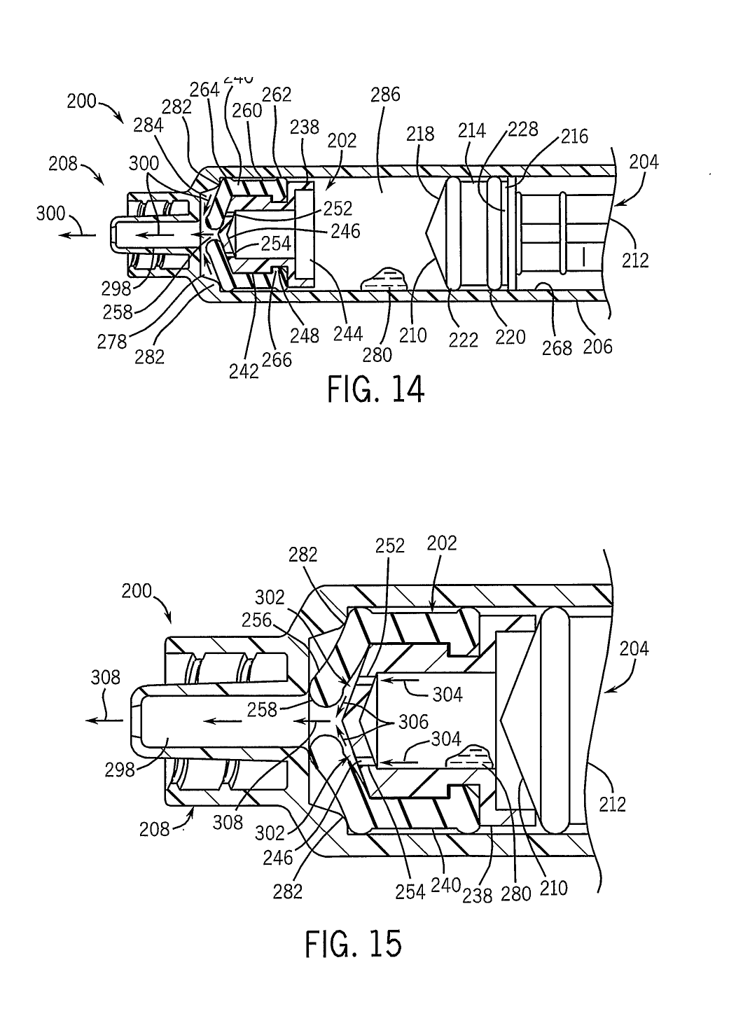

FIG. 14 is a partial cross-sectional view of the syringe 200 of FIG. 13, further illustrating a first injection of the medical fluid 278 from the syringe 200 immediately prior to an injection transition or intermediate position between multiple/sequential injections of the medical fluids 278, 280. The first injection of the medical fluid 278 is represented by arrows 300. Specifically, the illustrated syringe 200 can permit passage of the fist medical fluid 278 (e.g., a radiopharmaceutical or contrast agent) followed by the second medical fluid 280 (e.g., a biocompatible flush) through the central fluid flow passage 298 of the luer fitting 208 via the intermediate flow control plunger 202. In the illustrated embodiment, the intermediate flow control plunger 202 may be abutted against the flow control actuator 282 after discharging the first medical fluid 278. The first medical fluid 278 may be discharged from the first chamber 284 between the intermediate flow control plunger 202 and the second end 274 of the syringe barrel 206 by depressing the primary plunger 204 lengthwise along the axis 234. As the primary plunger 204 moves lengthwise along the syringe barrel 206, the flexible plunger sleeve 240 remains sealed against the fluid passage plunger insert 238 due to the pressure differential between the first and second chambers 284, 286. Upon reaching the flow control actuator 282, the intermediate flow control plunger 202 may become stationary to actuate the flexible plunger sleeve 240.

In other words, the flexible plunger sleeve 240 may remain closed or sealed with the fluid passage plunger insert 238 as long as the intermediate flow control plunger 202 is capable of moving in response to a pressure differential between the first and second cavities or chambers 284, 286. As such, the movement of the intermediate flow control plunger 202 maintains a fluid pressure balance between the first and second chambers 284, 286, such that the seal is maintained by the flexible plunger sleeve 240. When movement is no longer possible at the flow control actuator 282, the force or pressure of the second medical fluid 280 disposed in the second chamber 286 overcomes the flexible plunger sleeve 240 to enable discharge of the second medical fluid 280. At this stage, the primary plunger 204 moves lengthwise along the syringe barrel 206 while the intermediate flow control plunger 202 remains stationary.

FIG. 15 is a partial cross-sectional view of the syringe 200 of FIGS. 13-14, further illustrating actuation of the intermediate flow control plunger 202 at the flow control actuator 282. As illustrated, the flexible mouth portion 256 of the flexible plunger sleeve 240 is disposed at an offset away from the throat end 246 of the fluid passage plunger insert 238. In other words, a gap 302, may exist between the flexible mouth portion 256 and the throat end 246. In this generally unrestricted configuration, the second medical fluid 280 disposed between the primary plunger head 210 and the intermediate flow control plunger 202 may be forced through the passages 252, 254, the gap 302, and out through the central fluid flow passage 298 as illustrated by arrows 304, 306, and 308, respectively. In certain embodiments, as discussed above, the second medical fluid 280 may include a biocompatible flushing fluid, such as a heparin solution, sterilized water, a glucose solution, saline, or another suitable medical fluid. Accordingly, the second fluid injection or discharge may serve to substantially flush out or clean the various passages and interior portions of the syringe 200.

In certain embodiments, the syringes illustrated and described above with reference to FIGS. 1-15 may be filled or pre-filled with one or more medical fluids, such as contrast agents, radiopharmaceuticals, tagging agents, biocompatible flushes, or combinations thereof. For example, the disclosed syringes, e.g., 20, 110, 130, 170, and 200 may be filled or pre-filled with a first medical fluid in a first chamber and a second medical fluid in a second chamber. The first medical fluid may include a contrast agent for medical imaging, such as magnetic resonance imaging (MRI), computed tomography (CT), radiography (e.g., x-ray), or ultrasound. Alternatively, the first medical fluid may include a radioisotope or radiopharmaceutical for radiation-based treatment or medical imaging, such as positron emission tomography (PET) or single photon emission computed tomography (SPECT). In addition, the second medical fluid may include a biocompatible flush, such as heparin solution, sterilized water, glucose solution, saline, or another suitable substance. The disclosed syringes may be used to inject the first and second medical fluids one after another into a subject or patient. Alternatively, the disclosed multi-chamber, multi-stage, or sequential injection syringes, e.g., 20, 110, 130, 170, and 200 may be filled or pre-filled with a single medical fluid, such as a radiopharmaceutical or a contrast agent.

In certain embodiments, the subject (e.g., patient) may be scanned or generally imaged by a suitable medical diagnostic and/or imaging system, such as listed above. For example, after the radiopharmaceutical enters the blood stream and focuses on a particular organ or area of interest, the diagnostic and/or imaging system may function to acquire imaging data, process the data, and output one or more images. Thus, the diagnostic and/or imaging system may include detector/acquisition hardware and software, data/image processing hardware and software, data/image storage hardware and software, a display, a printer, a keyboard, a mouse, a computer workstation, a network, and other associated equipment.

FIG. 16 is a flowchart illustrating an embodiment of a method of use or syringe preparation process 350 utilizing one or more of the multi-chamber, multi-stage, or sequential injection syringes, e.g., 20, 110, 130, 170, and 200, of FIGS. 1-15. As illustrated, the process 350 may include filling a first chamber of a syringe with a first medical fluid (block 352). For example, the first medical fluid may include a radiopharmaceutical or a contrast agent. The process 350 may then include separating the first chamber from a second chamber of the syringe with an intermediate plunger having a flow-through check valve (block 354). For example, the intermediate plunger may include the intermediate flow control plunger 30 of FIGS. 1-12 or the intermediate flow control plunger 202 of FIGS. 13-15. The process 350 also may include filling the second chamber of the syringe with a second medical fluid (block 356). For example, the second medical fluid may include a biocompatible flush, such as heparin solution, sterilized water, glucose solution, saline, or another suitable substance. In addition, the process 350 may include closing the syringe with a primary plunger disposed about the second chamber opposite from the intermediate plunger (block 358).

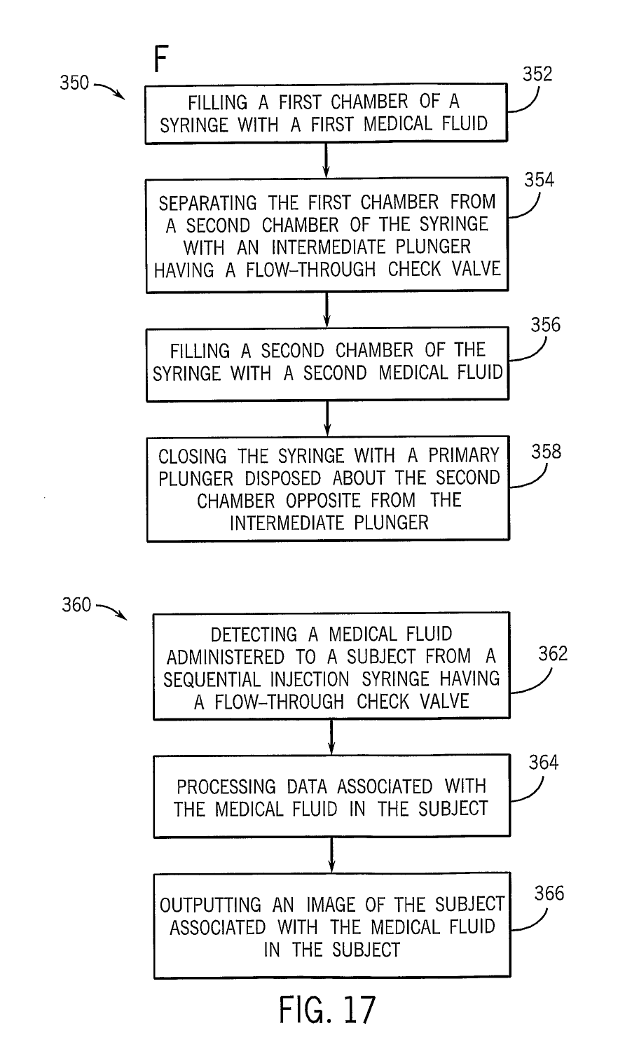

FIG. 17 is a flowchart illustrating an embodiment of a diagnostic imaging process 360 utilizing one or more of the multi-chamber, multi-stage, or sequential injection syringes, e.g., 20, 110, 130, 170, and 200, as illustrated in FIGS. 1-15. As illustrated, the process 360 may include detecting a medical fluid administered to a subject (e.g., a patient) from a sequential injection syringe having a flow-through check valve (block 362). The detection may include a variety of imaging modalities. The medical fluid may enable detection, or enhance detection, or tag a particular organ, or otherwise improve the imaging detection of a particular area of interest in the patient. For example, the syringe filled in the process 350 of FIG. 16 may be used to inject a subject with a radiopharmaceutical, a contrast agent, or another substance. By further example, one of the multi-chamber, multi-stage, or sequential injection syringes, e.g., 20, 110, 130, 170, and 200, as illustrated with reference to FIGS. 1-15 may be used to inject a radiopharmaceutical or a contrast agent into a subject. As discussed above, a contrast agent may be used for medical imaging, such as magnetic resonance imaging (MRI), computed tomography (CT), radiography (e.g., x-ray), or ultrasound. Alternatively, a radioisotope or radiopharmaceutical may be used for radiation-based treatment or medical imaging, such as positron emission tomography (PET) or single photon emission computed tomography (SPECT). At block 364, the process 360 may include processing data associated with the medical fluid in the subject. The process 360 also may include outputting an image of the subject associated with the medical fluid in the subject (block 366). Again, the foregoing procedures and resulting image directly benefit from the one or more medical fluids (e.g., radiopharmaceutical or contrast agent) administered with the multi-chamber, multi-stage, or sequential injection syringes, e.g., 20, 110, 130, 170, and 200, as illustrated and described with reference to FIGS. 1-15

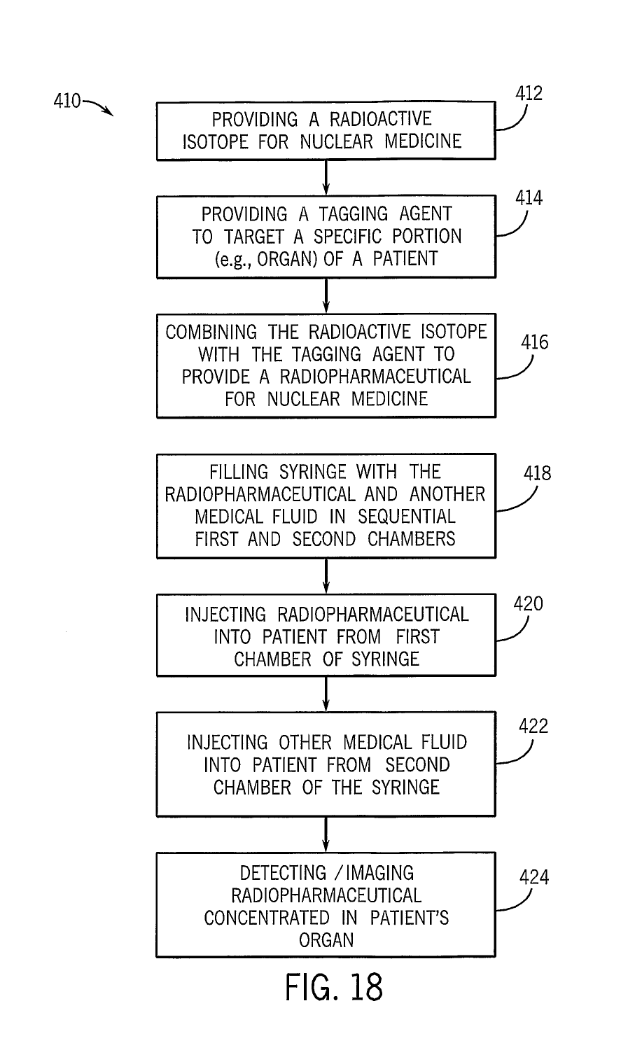

FIG. 18 is a flowchart illustrating an exemplary nuclear medicine process utilizing one or more of the multi-chamber, multi-stage, or sequential injection syringes, e.g., 20, 110, 130, 170, and 200, as illustrated with reference to FIGS. 1-15. As illustrated, the process 410 begins by providing a radioactive isotope for nuclear medicine at block 412. For example, block 412 may include eluting technetium-99m from a radioisotope generator as discussed in further detail below. At block 414, the process 410 proceeds by providing a tagging agent (e.g., an epitope or other appropriate biological directing moiety) adapted to target the radioisotope for a specific portion, e.g., an organ of a patient. At block 416, the process 410 then proceeds by combining the radioactive isotope with the tagging agent to provide a radiopharmaceutical for nuclear medicine. In certain embodiments, the radioactive isotope may have natural tendencies to concentrate toward a particular organ or tissue and, thus, the radioactive isotope may be characterized as a radiopharmaceutical without adding any supplemental tagging agent. At block 418, the process 410 may then involve filling a syringe with the radiopharmaceutical and another medical fluid in sequential first and second chambers, as discussed in detail above. For example, block 418 may include the process 350 of FIG. 16, and may include filling one of the multi-chamber, multi-stage, or sequential injection syringes, e.g., 20, 110, 130, 170, and 200, as illustrated with reference to FIGS. 1-15. At block 420, the process 410 then may proceed by injecting the radiopharmaceutical into a patient from the first chamber of the syringe. At block 422, the process 410 may continue by injecting the other medical fluid into the patient from the second chamber of the syringe. Again, the other fluid may include a biocompatible flush or another selected medical fluid. After a pre-selected time, the process 410 proceeds by detecting/imaging the radiopharmaceutical tagged to the patient's organ or tissue (block 424). For example, block 424 may include using a gamma camera or other radiographic imaging device to detect the radiopharmaceutical disposed on or in or bound to tissue of a brain, a heart, a liver, a tumor, a cancerous tissue, or various other organs or diseased tissue.

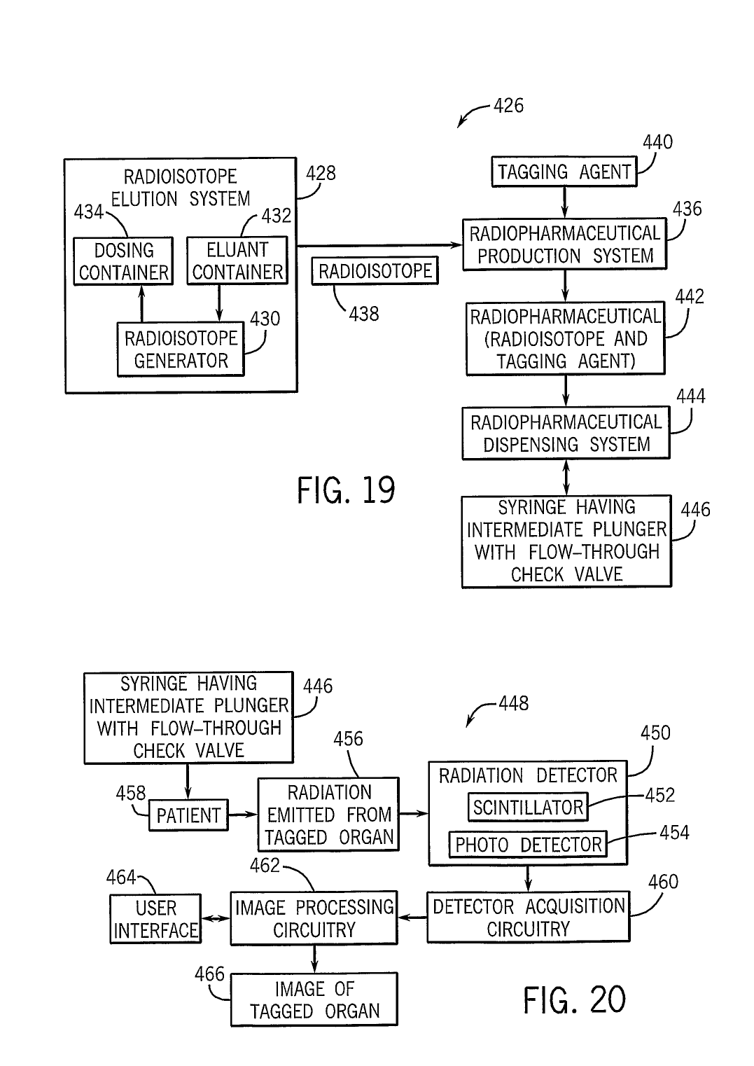

FIG. 19 is a block diagram of an exemplary system 426 for providing one or more of the multi-chamber, multi-stage, or sequential injection syringes, e.g., 20, 110, 130, 170, and 200, as illustrated in FIGS. 1-15 with one or more medical fluids (e.g., radiopharmaceutical and biocompatible flush) for use in a nuclear medicine application. As illustrated, the system 426 may include a radioisotope elution system 428 having a radioisotope generator 430, an eluant supply container 432, and an eluate output container or dosing container 434. In certain embodiments, the eluate output container 434 may be evacuated (in vacuum), such that the pressure differential between the eluant supply container 432 and the eluate output container 434 facilitates circulation of an eluant (e.g., saline) through the radioisotope generator 430 and out through an eluate conduit into the eluate output container 434. As the eluant (e.g., a saline solution) circulates through the radioisotope generator 430, the circulating eluant generally washes out or elutes a radioisotope (e.g., Technetium-99m). For example, one embodiment of the radioisotope generator 430 includes a radiation shielded outer casing (e.g., lead shell) that encloses a radioactive parent, such as molybdenum-99, adsorbed to the surfaces of beads of alumina or a resin exchange column. Inside the radioisotope generator 430, the parent molybdenum-99 transforms, with a half-life of about 67 hours, into metastable technetium-99m. The daughter radioisotope (e.g., technetium-99m) is generally held less tightly than the parent radioisotope (e.g., molybdenum-99) within the radioisotope generator 430. Accordingly, the daughter radioisotope can be extracted or washed out with a suitable eluant, such as an oxidant-free physiologic saline solution. The eluate output from the radioisotope generator 430 into the eluate output container 434 generally includes the eluant and the washed out or eluted radioisotope from within the radioisotope generator 430. Upon receiving the desired amount of eluate within the eluate output container 434, a valve may be closed to stop the eluant circulation and output of eluate. As discussed in further detail below, the extracted daughter radioisotope can then, if desired, be combined with a tagging agent to facilitate diagnosis or treatment of a patient (e.g., in a nuclear medicine facility).

As further illustrated in FIG. 19, the system 426 also includes a radiopharmaceutical production system 436, which functions to combine a radioisotope 438 (e.g., technetium-99m solution acquired through use of the radioisotope elution system 428) with a tagging agent 440. In some embodiment, this radiopharmaceutical production system 436 may refer to or include what are known in the art as “kits” (e.g., Technescan™ kit for preparation of a diagnostic radiopharmaceutical). Again, the tagging agent may include a variety of substances that are attracted to or targeted for a particular portion (e.g., organ, tissue, tumor, cancer, etc.) of the patient. As a result, the radiopharmaceutical production system 436 produces or may be utilized to produce a radiopharmaceutical including the radioisotope 438 and the tagging agent 440, as indicated by block 442. The illustrated system 426 may also include a radiopharmaceutical dispensing system 444, which facilitates extraction of the radiopharmaceutical into a syringe 446 having an intermediate plunger with a flow-through check valve. In the illustrated embodiment, the syringe may be one of the multi-chamber, multi-stage, or sequential injection syringes, e.g., 20, 110, 130, 170, and 200, as illustrated and described above with reference to FIGS. 1-15. Thus, the system 426 also may fill the syringe with an additional medical fluid, such as a biocompatible flush. For example, the multi-chamber, multi-stage, or sequential injection syringes, e.g., 20, 110, 130, 170, and 200, of FIGS. 1-15 may be filled with a radiopharmaceutical and a biocompatible flush in sequential chambers separated by the intermediate flow control plunger, e.g., 30 or 202. In certain embodiments, the various components and functions of the system 426 may be disposed within a radiopharmacy, which prepares the syringe 446 of the radiopharmaceutical for use in a nuclear medicine application. For example, the syringe 446 may be prepared and delivered to a medical facility for use in diagnosis or treatment of a patient.

FIG. 20 is a block diagram of an exemplary nuclear medicine imaging system 448 utilizing the multi-chamber, multi-stage, or sequential injection syringe 446 of radiopharmaceutical provided using the system 426 of FIG. 19. As illustrated, the nuclear medicine imagining system 448 includes a radiation detector 450 having a scintillator 452 and a photo detector 454. In response to radiation 456 emitted from a tagged organ within a patient 458, the scintillator 452 emits light that is sensed and converted to electronic signals by the photo detector 454. Although not illustrated, the imaging system 448 also can include a collimator to collimate the radiation 456 directed toward the radiation detector 450. The illustrated imaging system 448 also includes detector acquisition circuitry 460 and image processing circuitry 462. The detector acquisition circuitry 460 generally controls the acquisition of electronic signals from the radiation detector 450. The image processing circuitry 462 may be employed to process the electronic signals, execute examination protocols, and so forth. The illustrated imaging system 448 also includes a user interface 464 to facilitate user interaction with the image processing circuitry 462 and other components of the imaging system 448. As a result, the imaging system 448 produces an image 466 of the tagged organ within the patient 458. Again, the foregoing procedures and resulting image 466 directly benefit from the one or more medical fluids (e.g., radiopharmaceutical) administered with the multi-chamber, multi-stage, or sequential injection syringes, e.g., 20, 110, 130, 170, and 200, as illustrated and described with reference to FIGS. 1-15.

When introducing elements of various embodiments of the present invention, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top”, “bottom”, “above”, “below” and variations of these terms is made for convenience, but does not require any particular orientation of the components.

While the invention may be susceptible to various modifications and alternative forms, specific embodiments have been shown by way of example in the figures and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A syringe, comprising:
a barrel having an open end and a terminus disposed at an end opposite the open end, wherein the terminus has a passageway defined therein through which fluid may flow into and out of the barrel; and
a plunger disposed within the barrel between the open end and the terminus thereof, the plunger comprising a one-way valve having a fluid passage defined therein through an interior of the plunger from an upstream side of the plunger to a downstream side of the plunger.

2. The syringe of claim 1, wherein the plunger is disposed between first and second chambers in the syringe.

3. The syringe of claim 2, comprising another plunger disposed adjacent the second chamber opposite from the plunger.

4. The syringe of claim 1, wherein the plunger comprises a shaft coupled to a plunger head.

5. The syringe of claim 4, wherein the one-way valve is disposed in the plunger head.

6. (canceled)

7. The syringe of claim 1, wherein the plunger comprises a substantially rigid first component and a substantially elastomeric second component that movably engage one another in an open position and a closed position relative to the fluid passage.

8. The syringe of claim 1, wherein the plunger comprises a plurality of concentric members, including an outer member and an inner member, and the fluid passage is disposed inside the outer member.

9. The syringe of claim 1, wherein the plunger comprises a substantially resilient sleeve disposed about a substantially rigid core.

10. The syringe of claim 9, wherein the substantially resilient sleeve comprises a throat.

11. The syringe of claim 9, wherein the fluid passage of the plunger is disposed between the substantially resilient sleeve and the substantially rigid core.

12. The syringe of claim 9, wherein the substantially rigid core comprises the fluid passage.

13. The syringe of claim 1, wherein the fluid passage comprises a first passage offset from a second passage, wherein the first passage is disposed in a substantially rigid portion of the plunger, and the second passage is disposed in a substantially flexible portion of the plunger.

14. The syringe of claim 1, wherein the fluid passage comprises a plurality of passages disposed in a perforated structure.

15. The syringe of claim 1, wherein the syringe comprises medical fluids disposed on opposite sides of the plunger.

16. The syringe of claim 1, comprising a fill port disposed in the syringe.

17. The syringe of claim 16, wherein the fill port is disposed in the plunger.

18. The syringe of claim 16, wherein the fill port is disposed in a barrel of the syringe, and the plunger is disposed inside the syringe.

19. The syringe of claim 1, comprising a radioisotope generator, a fluid dispensing system, a power injector, a support structure, a rotatable arm, a stand, an electronic control unit, a computer, an imaging system, a diagnostic system, or a combination thereof coupled to or generally associated with the syringe.

20-25. (canceled)

26. The syringe of claim 1, wherein the syringe is designed to be mounted on a power injector that a drive ram of the power injector can move the plunger of the syringe relative to the barrel of the syringe to expel fluid from within the syringe.

27. (canceled)

28. A syringe, comprising:
a barrel having an open end and a terminus disposed at an end opposite the open end, wherein the terminus has a passageway defined therein through which fluid may flow into and out of the barrel;
a plunger disposed within the barrel between the open end and the terminus thereof, the plunger comprising a check valve; and
a check-valve actuator disposed inside the barrel near the terminus of the barrel.

29. The syringe of claim 28, wherein the check-valve actuator comprises an inwardly protruding portion inside the barrel at the terminus.

30. The syringe of claim 29, wherein the inwardly protruding portion comprises a generally conical geometry.

31. The syringe of claim 29, wherein the inwardly protruding portion comprises an annular geometry.

32-40. (canceled)

41. A method of using a syringe, comprising:
biasing a plunger of a syringe toward a terminus of the syringe to discharge a first medical fluid from between the terminus and an intermediate plunger of the syringe;
contacting the terminus of the syringe with the intermediate plunger; and
discharging a second medical fluid from the syringe through the intermediate plunger while the terminus and the intermediate plunger are in contact.
